(19) United States
(12) Patent Application Publication
(10) Pub. No.: US 2006/0105052 A1
(43) Pub. Date: May 18, 2006

(54) Cationic nanoparticle having an inorganic core

(76) Inventors: Havva Yagci Acar, Sariyer-Istanbul (TR); Andrew Soliz Torres, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 10/989,632

(22) Filed: Nov. 15, 2004

(51) Int. Cl. A61K 48/00; C12N 15/87; A61K 9/50

(52) U.S. Cl. 424/490; 435/459; 977/916

(57) ABSTRACT

A cationic nanoparticle having an inorganic core and at least one outer cationic coating is described. The at least one outer cationic coating substantially covers the inorganic core and has at least one organo-silane. The organo-silane includes:
—Si(R¹)₃
wherein R¹independently at each occurrence is an alkoxy group, a hydroxyl group, a halide, an alkyl group, or hydrogen, and wherein at least one R¹ of the three R¹s is not an alkyl group. A nanocomplex having a cationic nanoparticle and at least one oligonucleotide attached to the cationic nanoparticle is also described. Methods of making cationic nanoparticles and nanocomplexes are also described. Also described are methods of delivering an oligonucleotide into a cell in-vitro, to a subject in-vivo, and monitoring the delivery of an oligonucleotide.

### BACKGROUND OF INVENTION

The invention relates to a cationic nanoparticle having an inorganic core with at least one cationic coating substantially covering the inorganic core. More particularly, the invention relates to a cationic nanoparticle with an organo-silane cationic coating and capable of attaching to an oligonucleotide and method of making and using the same.

Nanotechnology, relating particularly to cationic nanoparticles, is useful in a number of fields, such as diagnostic medicine, molecular imaging, and as delivery agents or carriers, such as delivering oligonucleotides to a cell in-vitro or to a subject in-vivo. There are currently 2 ways of delivering oligonucleotides: viral and non-viral delivery. Regarding viral delivery, viral delivery often causes cytotoxicity. Regarding non-viral delivery, transfection efficiency is often poor for various reasons. Some non-viral delivery systems are based on agglomerates of magnetic particles and gene-vectors which result in large particle sizes, such as from about 100 nm to 1 micron. Non-viral vectors generally have large particle sizes, such as from about 100 nm to 1 micron. These large particle sizes result in weak gene-delivery to the tissue of interest because of size-restricted diffusion and rapid blood clearance.

The cationic nanoparticles obtained by the current methods are agglomerates. When such agglomeration occurs, the efficacy of the cationic nanoparticles in a given application is lost. Therefore, what is needed is a cationic nanoparticle resistant to agglomeration. A need also exists for cationic nanoparticles that are not cytotoxic. Also needed are cationic nanoparticles that can attach to an oligonucleotide. Also needed are non-agglomorated cationic nanoparticle-oligonucleotide complexes. Also needed are cationic nanoparticles that can effectively deliver an oligonucleotide into a cell in-vitro or to a subject in-vivo.

### SUMMARY OF INVENTION

The present invention meets these and other needs by providing a cationic nanoparticle comprising an inorganic core and at least one outer cationic coating.

Accordingly, one aspect of the invention is to provide a cationic nanoparticle. The cationic nanoparticle comprises an inorganic core and at least one outer cationic coating substantially covering the inorganic core. The at least one outer cationic coating comprises at least one organo-silane, wherein the at least one organo-silane comprises:
—Si(R¹)₃.
R¹ independently at each occurrence is an alkoxy group, a hydroxyl group, a halide, an alkyl group, or hydrogen, and wherein at least one R¹ of the three R¹s is not an alkyl group.

A second aspect of the invention is to provide a nanocomplex comprising a cationic nanoparticle and at least one oligonucleotide attached to the cationic nanoparticle; and wherein the nanocomplex is substantially unagglomerated. The cationic nanoparticle comprises an inorganic core and at least one outer cationic coating substantially covering the inorganic core. The at least one outer cationic coating comprises at least one organo-silane, wherein the at least one organo-silane comprises:
—Si(R¹)₃.
R¹ independently at each occurrence is an alkoxy group, a hydroxyl group, a halide, an alkyl group, or hydrogen, and wherein at least one R¹ of the three R¹s is not an alkyl group.

A third aspect of the invention is to provide a method of making a plurality of cationic nanoparticles, wherein each cationic nanoparticle comprises an inorganic core and at least one outer cationic coating substantially covering the inorganic core. The at least one outer cationic coating comprises at least one organo-silane, wherein the at least one organo-silane comprises:
—Si(R¹)₃.
R¹ independently at each occurrence is an alkoxy group, a hydroxyl group, a halide, an alkyl group, or hydrogen, and wherein at least one R¹ of the three R¹s is not an alkyl group. The method comprises the steps of: providing an aqueous solution comprising metal ions; heating the aqueous solution comprising the metal ions; providing a base and at least one cationic coating material to the aqueous solution, wherein the at least one cationic coating material comprises at least one organo-silane, wherein the at least one organo-silane comprises:
—Si(R¹)₃.
wherein R¹ independently at each occurrence is an alkoxy group, a hydroxyl group, a halide, an alkyl group, or hydrogen, wherein at least one R¹ of the three R¹s is not an alkyl group, and wherein the base reacts with the metal ions to form the inorganic core and wherein the base reacts with the at least one cationic coating material to substantially cover the inorganic core to form the plurality of cationic nanoparticles; and optionally protonating the at least one outer cationic coating of the formed cationic nanoparticle by adjusting the aqueous solution to a pH in a range from about 2 to about 9.

A fourth aspect of the invention is to provide a method of making a plurality of nanocomplexes wherein each nanocomplex comprises a cationic nanoparticle and at least one oligonucleotide attached to the cationic nanoparticle; and wherein the nanocomplex is substantially unagglomerated. The cationic nanoparticle comprises an inorganic core and at least one outer cationic coating substantially covering the inorganic core. The at least one outer cationic coating comprises at least one organo-silane, wherein the at least one organo-silane comprises:
—Si(R¹)₃.
R¹ independently at each occurrence is an alkoxy group, a hydroxyl group, a halide, an alkyl group, or hydrogen, and wherein at least one R¹ of the three R¹s is not an alkyl group. The method comprises the steps of: providing a plurality of cationic nanoparticles and a plurality of oligonucleotides into an aqueous solution; and attaching the at least one oligonucleotide to the at least one cationic nanoparticle to form the plurality of the nanocomplexes.

A fifth aspect of the invention is to provide a method of delivering at least one oligonucleotide into a cell. The method comprises providing at least one nanocomplex into a solution of cells. The nanocomplex comprises a cationic nanoparticle and at least one oligonucleotide attached to the cationic nanoparticle. The nanocomplex is substantially unagglomerated. The cationic nanoparticle comprises an inorganic core and at least one outer cationic coating substantially covering the inorganic core. The at least one outer cationic coating comprises at least one organo-silane, wherein the at least one organo-silane comprises:
—Si(R¹)₃.
R¹ independently at each occurrence is an alkoxy group, a hydroxyl group, a halide, an alkyl group, or hydrogen, and wherein at least one R¹ of the three R¹s is not an alkyl group.

A sixth aspect of the invention is to provide a method of delivering at least one oligonucleotide to a subject. The method comprises administering at least one nanocomplex to the subject. The nanocomplex comprises a cationic nanoparticle and at least one oligonucleotide attached to the cationic nanoparticle, and is substantially unagglomerated. The cationic nanoparticle comprises an inorganic core and at least one outer cationic coating substantially covering the inorganic core. The at least one outer cationic coating comprises at least one organo-silane, wherein the at least one organo-silane comprises:
—Si(R¹)₃.
R¹ independently at each occurrence is an alkoxy group, a hydroxyl group, a halide, an alkyl group, or hydrogen, wherein at least one R¹ of the three R¹s is not an alkyl group.

A seventh aspect of the invention is to provide a method of monitoring the delivery of at least one oligonucleotide to a subject. The method comprises the steps of: administering at least one nanocomplex to a subject; obtaining a magnetic resonance image of the subject to achieve a signal of the concentration of the at least one nanocomplex administered to the subject; and correlating the signal of the at least one nanocomplex to the concentration of the at least one oligonucleotide administered to the subject. The nanocomplex comprises a cationic nanoparticle and at least one oligonucleotide attached to the cationic nanoparticle; and wherein the nanocomplex is substantially unagglomerated. The cationic nanoparticle comprises an inorganic core and at least one outer cationic coating substantially covering the inorganic core. The at least one outer cationic coating comprises at least one organo-silane, wherein the at least one organo-silane comprises:
—Si(R¹)₃.
R¹ independently at each occurrence is an alkoxy group, a hydroxyl group, a halide, an alkyl group, or hydrogen, wherein at least one R¹ of the three R¹s is not an alkyl group.

These and other aspects, advantages, and salient features of the present invention will become apparent from the following detailed description, the accompanying drawings, and the appended claims.

### BRIEF DESCRIPTION OF DRAWINGS

FIG. 1 is a schematic representation of a cationic nanoparticle of one embodiment of the invention;

FIG. 2 is a transmission electron microscopic image (TEM) of N-trimethoxysilylpropyl N,N,N,-tri-methylammonium chloride coated cationic nanoparticles of one embodiment of the invention;

FIG. 3 is a TEM of N-[3-(trimethoxysilyl)propyl] modified polyethyleneimine coated cationic nanoparticles of one embodiment of the invention;

FIG. 4 is a characteristic magnetization curve plotted as a function of magnetic field;

FIG. 5 is a schematic representation of a nanocomplex of one embodiment of the invention;

FIG. 6a is an agarose gel showing the presence of oligonucleotides in a nanocomplex of one embodiment of the invention;

FIG. 6b is the same gel as that shown in FIG. 6a Perl stained;

FIG. 7a is an agarose gel showing the stability of nanocomplexes of one embodiment of the invention in the presence of serum;

FIG. 7b is the agarose gel as that shown in FIG. 7a Perl stained;

FIG. 8 is a flow diagram showing a method of making a plurality of cationic nanoparticles of one embodiment of the invention;

FIG. 9 is a schematic representation of a method of making a plurality of nanocomplexes of one embodiment of the invention;

FIG. 10a is a bright field microscopic image of mouse macrophages fixed and Perl-stained that are not incubated with nanocomplexes of one embodiment of the invention;

FIG. 10b is a bright field microscopic image showing the presence of nanocomplexes into cells after administering the cells with nanocomplexes for 24 hours;

FIG. 11a are bright field confocal microscopy images of cells after 6-hour incubation with cationic nanoparticles that are not attached to an oligonucleotide;

FIG. 11b are laser excited fluorophore images of the cells shown in FIG. 11a;

FIG. 11c are bright field confocal microscopy images of cells after 6-hour incubation with a nanocomplex of cationic nanoparticles that are attached to a fluorescent tagged oligonucleotide;

FIG. 11d are laser excited fluorophore images of the same cells as shown in FIG. 11c;

FIG. 12 are RT-PCR (reverse transcription polymerase chain reaction) analyses showing that incubating cells with nanocomplexes deliver active oligonucleotides into cells;

FIG. 13a is an in-vivo magnetic resonance image (MRI) of a rat liver that has not been injected with a nanocomplex of one embodiment of the invention;

FIG. 13b is an in-vivo MRI of rat liver 24 hours after being injected with a nanocomplex; and

FIG. 13c is an in-vivo MRI of rat liver 24 hours after being injected with a nanocomplex.

### DETAILED DESCRIPTION

In the following description, like reference characters designate like or corresponding parts throughout the several views shown in the figures. It is also understood that terms, such as “top”, “bottom”, “outward”, “inward”, and the like are words of convenience and are not to be construed as limiting terms. Whenever a particular aspect of the invention is said to comprise or consist of at least one element of a group and combinations thereof, it is understood that the aspect may comprise or consist of any of the elements of the group, either individually or in combination with any of the other elements of that group.

Referring to the drawings in general, it will be understood that the illustrations are for the purpose of describing a particular embodiment of the invention and are not intended to limit the invention thereto.

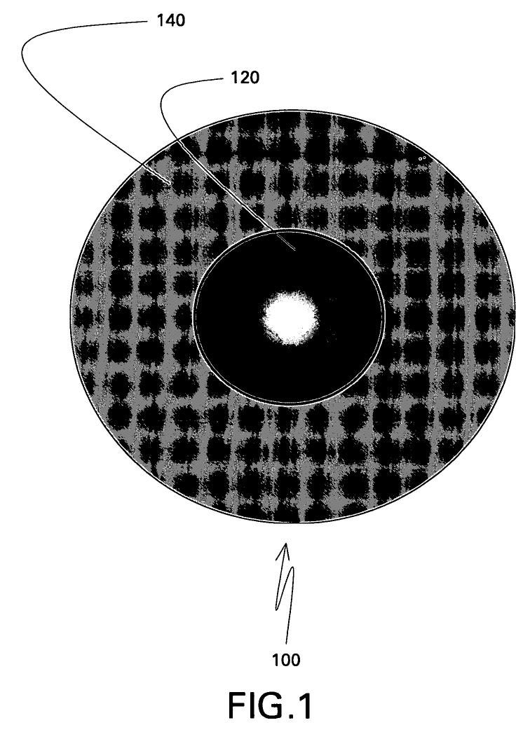

A schematic representation of a cross-sectional view of a cationic nanoparticle of the present invention is shown in FIG. 1. The cationic nanoparticle 100 comprises an inorganic core 120 and at least one outer cationic coating 140. The outer cationic coating substantially covers the inorganic core 120.

In one embodiment, the inorganic core 120 is a substantially crystalline inorganic material. In this context, “substantially crystalline” is understood to mean that inorganic core 120 comprises at least 50 volume percent and, preferably, at least 75 volume percent, crystalline material. In one particular embodiment, the inorganic core 120 is substantially monodisperse. Monodisperse means the cores are of a similar size, based on about a 25% to 30% standard deviation.

The inorganic core 120 may comprise a variety of inorganic materials, including, but not limited to, transition metals in elemental form, metal oxides, and superparamagnetic materials that are known in the art. The inorganic material may comprise any of the materials mentioned above, either individually or any combination thereof. In one embodiment, the inorganic core 120 is magnetic. In a particular embodiment, the magnetic inorganic core 120 comprises iron oxide. The iron oxide may comprise at least one of magnetite, maghemite, or a combination thereof. In a particular embodiment, the inorganic core 120 is superparamagnetic.

In one embodiment, the cationic nanoparticle 100 is spherical and has a diameter in a range from about 1 nm to about 100 nm. In another embodiment, the cationic nanoparticle 100 has a diameter in a range from about 5 nm to about 60 nm. In yet another embodiment, the cationic nanoparticle 100 has a diameter in a range from about 5 nm to about 20 nm. In a particular embodiment, a plurality of cationic nanoparticles 100 is substantially unagglomerated. Substantially unagglomerated means the cationic nanoparticle-to-cationic nanoparticle contact is minimal such that a cationic nanoparticle 100 has a diameter less than about 100 nm as measured by dynamic light scattering. Use of the word diameter does not restrict the cationic nanoparticles 100 to spherical shapes.

The cationic coating 140 means the coating carries a positive electrical charge that is counterbalanced by ions of negative charges in solution. The cationic coating 140 may contain chemical groups that can ionize to produce a positively charged coating or may contain chemical groups that preferentially adsorb negatively charged ions or species. A Zeta potential describes the nature of the electrostatic potential near the surface of a particle, therefore indicating the anionic, cationic or neutral nature of the particle. A positive Zeta potential demonstrates the cationic nature of the cationic nanoparticle 100. In one embodiment, the cationic nanoparticle 100 has a Zeta potential of 30-40 m.

The outer cationic coating 140 creates a charge repulsion between cationic nanoparticles 100, inhibiting a cationic nanoparticle 100 from contacting an adjacent cationic nanoparticle 100, thereby preventing a plurality of such cationic nanoparticles 100 from agglomerating. In one embodiment, the at least one outer cationic coating 140 has a thickness in a range from about 1 nm to about 50 nm. In another embodiment, the at least one outer cationic coating 140 has thickness in a range from about 1.5 nm to about 3 nm.

The at least one outer cationic coating 140 comprises at least one organo-silane. The at least one organo-silane comprises:
—Si(R¹)₃,
where R¹ independently at each occurrence is an alkoxy group, a hydroxyl group, a halide, an alkyl group, or hydrogen, and wherein at least one R¹ of the three R¹s is not an alkyl group. Whenever the term “halide” is used, “halide” includes halides as well as halogens unless noted otherwise. Also, the outer cationic coating 140 may comprise a plurality of the organo-silanes.

In one example, the at least one outer cationic coating 140 comprises:
Xₙ—R—Si(R¹ )₃,
where R¹ is as previously described and at least one R¹ of the three R¹s is not an alkyl group. R independently, at each occurrence, is an alkyl group or an aryl group. X independently, at each occurrence, is NH₂, NHR², NR²R³, or a water-soluble biocompatible cationic polymer; and n is an integer in a range from 1 to about 3.

In another example, the at least one outer cationic coating 140 comprises a water-soluble biocompatible cationic polymer comprising repeat units. In one embodiment, some of the repeat units have the following structure: 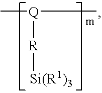
where R¹ is as previously described. R, independently, at each occurrence, is either an alkylene group or an arylene group. M is an integer greater than or equal to at least 1; Q, independently at each occurrence, is either an aliphatic radical or cycloaliphatic radical. The water-soluble biocompatible cationic polymer comprises a finite number of repeat units.

Non-limiting examples of the at least one outer cationic coating 140 include at least one of an organo-silane modified polyethylenimine, an organo-silane modified polyethylenimine, an organo-silane modified poly(lysine), an organo-silane modified poly(aspargine), an organo-silane modified chitosane, an organo-silane modified poly(L-omithine), an organo-silane modified poly(vinylamine), an organo-silane modified poly(amido amine), N-trimethoxysilylpropyl-N,N,N,-tri-methyl-ammonium, N-(trimethoxysilylethyl)benzyl-N,N,N-trimethylammonium chloride, N-(2-aminoethyl)-3-aminopropyltrimethoxysilane, a 3-aminopropyltrimethoxysilane, an aminopropylsilanetriol, and combinations thereof. “Organo-silane modified” means comprising at least one —Si(R¹)₃ organo-silane as described hereinabove. The at least one outer cationic coating 140 may comprise any such organo-silanes, either individually or in any combination thereof. In one embodiment, the outer cationic coating 140 may comprise a plurality of the organo-silanes wherein the plurality of organo-silanes may comprise a single type of the organo-silane or various types of the organo-silane.

In one embodiment, the at least one outer cationic coating 140 comprises N-trimethoxysilylpropyl N,N,N,-tri-methylammonium:

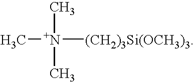

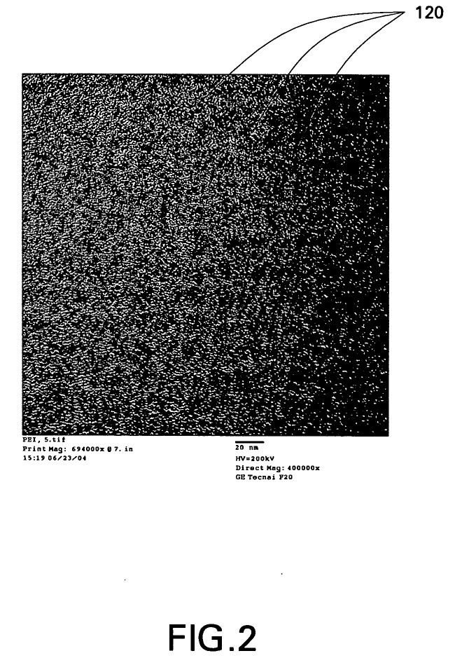

FIG. 2 is a transmission electron microscopic (TEM) image) of a cationic nanoparticles 100 with an outer cationic coating 140 comprising N-trimethoxysilylpropyl N,N,N,-tri-methylammonium chloride. The TEM image shows that the inorganic cores 120 are substantially monodisperse and that the cationic nanoparticles 100 are substantially unagglomerated. The TEM image also shows that the inorganic cores 120 have sizes in a range from 2 nm to 10 nm. The unagglomerated and nanoscale size of the cationic nanoparticles 100 makes the cationic nanoparticles 100 suitable for various applications, such as magnetic resonance imaging, transfection, drug delivery, and cell tracking.

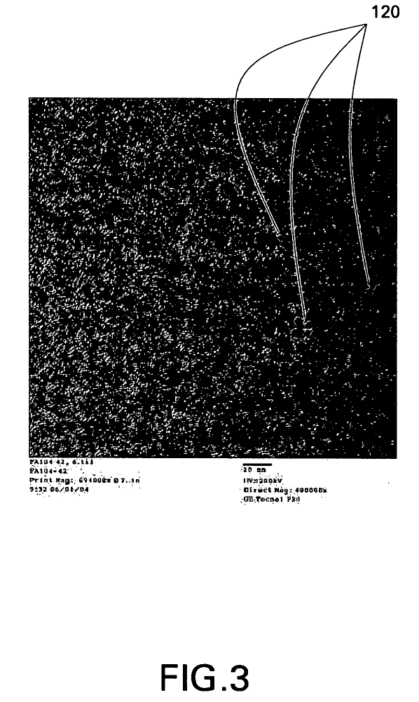

In another embodiment, the at least one outer cationic coating 140 comprises an organo-silane modified polyethyleneimine. FIG. 3 is a transmission electron microscopic image of a cationic nanoparticle 100 with an outer cationic coating 140 comprising N-[3-(trimethoxysilyl)propyl] polyethyleneimine hydrochloride. The TEM image shows that the inorganic cores 120 are substantially monodisperse and that the cationic nanoparticles 100 are substantially unagglomerated. In one example of the organo-silane modified polyethyleneimine cationic coating 140, the —Si(R¹)₃ organo-silane comprises trimethoxysilyl. An example of trimethoxysilyl is N-[3-(trimethoxysilyl)propyl] with propyl as a linker. In a particular example of when the organo-silane modified polyethyleneimine cationic coating 140 comprises trimethoxysily, the organo-silane modified polyethyleneimine has a molecular weight of less than about 25,000. In another example, the organo-silane modified polyethyleneimine may have a molecular weight of less than about 2,000 Da. In yet another example, the organo-silane modified polyethyleneimine has a molecular weight in a range from about 500 Da to about 2,000 Da and the organo-silane comprises about 10% by weight of the outer cationic coating 140. In one embodiment, the —Si(R¹)₃ organo-silane, such as trimethoxysily, comprises from about 10% to about 60% by weight of the outer cationic coating 140. In another embodiment, the organo-silane comprises from about 10% to about 40% by weight of the outer cationic coating 140. In yet another embodiment, the organo-silane comprises about 10% by weight of the outer cationic coating 140.

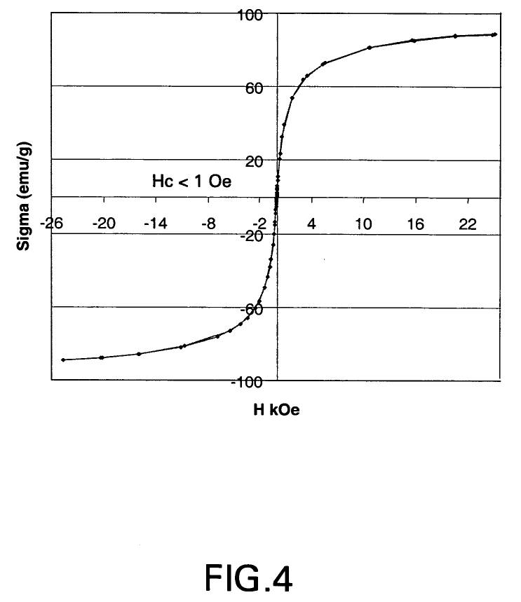

FIG. 4 is a characteristic magnetization curve plotted as a function of magnetic field. The behavior of the magnetic field is indicative of the superparamagnetic nature of the cationic nanoparticles 100. The cationic nanoparticles 100 exhibit a magnetic moment in the presence of a magnetic field. When the magnetic field is removed, the magnetization is lost.

Table 1 below shows some characteristics of the cationic nanoparticle 100 with two different outer cationic coatings 140: N-trimethoxysilylpropyl N,N,N,-tri-methylammonium chloride and N-[3-trimethoxysilyl)propyl] polyethyleneimine hydrochloride.

In magnetic resonance imaging (MRI), an image of an organ or tissue is obtained by placing a subject in a strong external magnetic field and observing protons present in the subject's organs or tissues after excitation by a radio frequency magnetic field. The proton relaxation times, termed as R1 (longitudinal relaxation time) and R2 (transverse relaxation time) depend on the chemical and physical environment of the organ or tissue water protons. Both R1 and R2 vary from tissue to tissue and strongly affect MR image intensity. To generate an MR image having good contrast, either one of R1 or R2 of the tissue to be imaged must be different from R1 or R2 of background tissue. One way of improving the contrast of MR images is to use a MRI contrast agent. R2/R1 ratio indicates the type of contrast with which the MRI contrast agent will be most effective.

The Msat (Saturation magnetization) is the amount of magnetic field that a magnet can produce. Strong magnets have higher saturation.

TABLE 1

| | Zeta | | | | | |
|---|---|---|---|---|---|---|
| | poten- | | Msat | | | |
| | tial | Size | (emu/ | R1 | R2 | |
| Coating | (mV) | (nm) | g) | (/mM/s) | (/mM/s) | R2/R1 |
| Trimethoxysilyl- | 30-40 | 54 | 41 | 4.25 | 28.08 | 6.602 |
| propylmodified | | | | | | |
| polyethyleneimine | | | | | | |
| N-trimethoxy- | 36 | 15 | 88 | 11.84 | 63.75 | 5.38 |
| silylpropyl | | | | | | |
| N,N,N,-tri- | | | | | | |
| methylammonium | | | | | | |
| chloride | | | | | | |

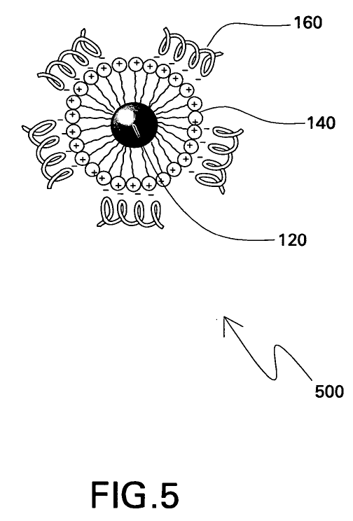

The cationic nature of the cationic nanoparticles 100 provides the nanoparticles 100 with various advantages. For example, the cationic nature of nanoparticles 100 allows the nanoparticles 100 to ionically attach to negatively charged species, such as oligonucleotides, or to alter biodistribution. Consequently, another aspect of the invention is to provide a nanocomplex 500 comprising the cationic nanoparticle 100 as described hereinabove and at least one oligonucleotide 160 attached to the cationic nanoparticle 100. FIG. 5 is a schematic representation of a nanocomplex 500.

The at least one oligonucleotide 160 may be single or double-stranded, linear or circular, natural or synthetic, and without any size limitation. The oligonucleotide may be in the form of a plasmid or of viral DNA or RNA. Furthermore, the oligonucleotide may include modifications, such as phosphothioates or peptide nucleic acids (PNA).

The at least one oligonucleotide 160 comprises at least one of a DNA molecule, an RNA molecule, and combinations thereof, and may comprise any such individual DNA, RNA, or any combination thereof. In one embodiment, the oligonucleotide comprises a plurality of oligonucleotides, wherein each of the oligonucleotides may independently either be an RNA molecule, DNA molecule, or any combination thereof. In one embodiment, the oligonucleotide 160 comprises at least one RNA. The RNA comprises at least one of a short inhibitory RNA, a short hairpin RNA, a micro RNA, either individually or in any combination. In one embodiment, the at least one RNA comprises a plurality of RNA, wherein each of the RNA independently is any such RNA molecule. In one embodiment, the RNA comprises short inhibitory (siRNA). In one example, the siRNA may comprise less than about 100 base pairs. In another example, the siRNA may comprise less than about 40 base pairs. In yet another example, the siRNA may comprise less than about 24 base pairs. One embodiment of siRNA is mature duplex siRNA. The double-stranded mature duplex siRNA may be formed by a single self-complementary RNA strand or two complementary RNA strands. RNA duplex formation may be initiated either inside or outside the cell. The RNA may be introduced in an amount which allows delivery of at least one copy per cell.

The at least one oligonucleotide 160 is attached to the cationic nanoparticle 100. In one embodiment, the oligonucleotide 160 may be attached to the cationic nanoparticle by ionic interaction. The oligonucleotide 160 attaches to the cationic nanoparticle 100 as the negatively charged oligonucleotide 160 ionically interacts with the positively charged cationic coating 140. Furthermore, the oligonucleotide 160 may attach to the cationic nanoparticle 100 at a plurality of sites on the positively charged cationic coating 140. Also, a plurality of oligonucleotides 160 may attach to the cationic nanoparticle 100. Each of the oligonucleotides 160 may independently attach to the cationic nanoparticle 100 at different sites and in different orientations.

In one embodiment, a plurality of nanocomplexes 500 is substantially unagglomerated. A substantially unagglomerated nanocomplex 500 means that the nanocomplex 500 has a size less than about 100 nm and is formed by the ionic interaction between the cationic nanoparticle 100 and at least one oligonucleotide 160 and the ionic interaction does not substantially change the size of the cationic nanoparticle 100, as measured by dynamic light scattering.

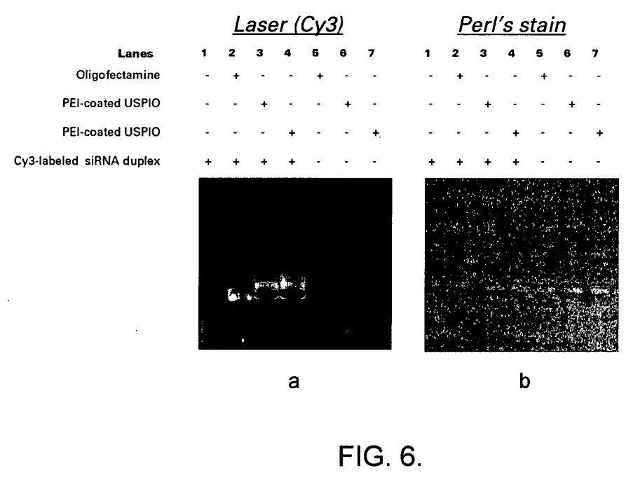

FIG. 6a is an agarose gel showing the presence of oligonucleotides 160 in a nanocomplex 500. Lanes 3-4 have cationic nanoparticles 100 attached to fluorescent-labeled oligonucleotide 160. USPIO means ultra small superparamagnetic iron oxide cationic nanoparticles 100. The cationic nanoparticles 100 comprise an organo-silane modified polyethyleneimine cationic coating 140. The fluorescent label is Cy3 and the oligonucleotide comprise siRNA duplex. In contrast, lanes 6-7 have cationic nanoparticles 100 that are not attached to any oligonucleotides. Samples containing Oligofectamine, a commercially available transfector reagent which binds oligonucleotides, attached to fluorescent-labeled oligonucleotide (lane 2) and Oligofectamine alone (lane 5) are shown as a control. A sample of free fluorescent-labeled oligonucleotide (Cy3-labeled siRNA duplex) is shown in lane 1. The images in FIG. 6a were obtained using a Biorad Molecular Imaging system having laser and filter inputs optimized for Cy3 fluorescence

FIG. 6b is the same gel as that shown in FIG. 6a Perl stained to show the presence of iron. The presence of iron confirms the presence of the inorganic core 120. The iron in the inorganic cores 120 of the cationic nanoparticles 100 is seen in lanes 3-4. FIG. 6a and 6b combined show the nanocomplex of the present invention with both a cationic nanoparticle 100 and an oligonucleotide 160 attached to the cationic nanoparticle 100 by showing both the presence of the oligonucleotide (6a) in lanes 3-4 as well as the presence of the cationic nanoparticle (6b) in lanes 3-4.

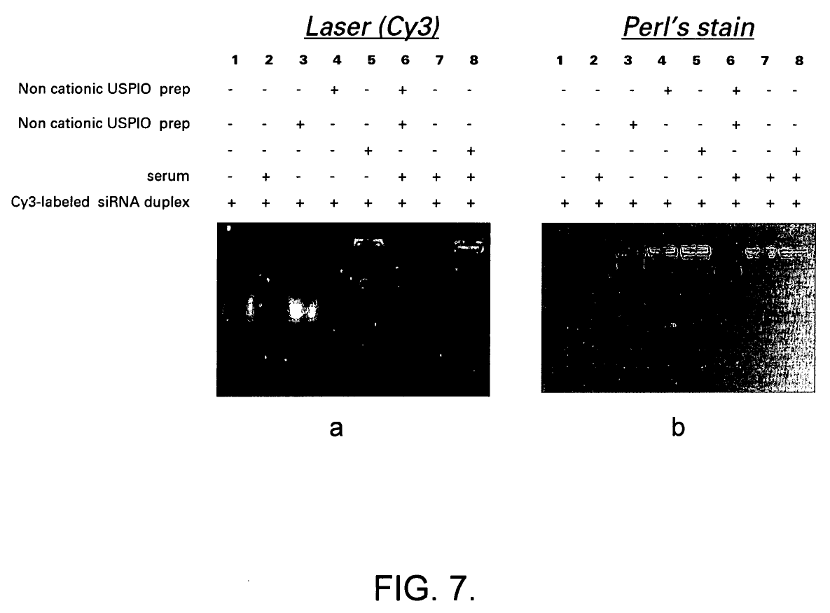

FIG. 7a is an agarose gel, similar to FIG. 6a, showing that the nanocomplex 500 does not degrade in the presence of serum. A nanocomplex 500 that does not degrade in the presence of serum may be desirable because serum contains abundant nucleases which can destroy oligonucleotides 160. For in-vivo delivery of active oligonucleotides 160, the oligonucleotides 160 must be delivered intact. The cationic nanoparticles 100 attached to oligonucleotides 160 (Cy3-labeled siRNA duplex) in the absence of serum are in lane 5. Cationic nanoparticles 100 attached to oligonucleotides 160 in presence of serum are in lane 8. The cationic nanoparticles 100 comprise a silane modified polyethyleneimine cationic coating 140. Samples containing non-cationic nanoparticles with labeled oligonucleotides in the absence (lanes 3-4) or presence (lanes 6-7) of serum are shown as controls. A sample of “free” fluorescent-labeled oligonucleotides 160 (Cy3-labeled siRNA duplex) is shown in lane 1 in the absence of serum and in lane 2 in the presence of serum. “Free” means not attached to a cationic nanoparticle 100. Images in FIG. 7a were obtained using a Biorad Molecular Imaging system using laser and filter inputs optimized for Cy3 fluorescence. FIG. 7b is the same gel as that shown in FIG. 7a and is Perl stained to indicate the location of iron, thereby confirming the presence and location of the inorganic core 120.

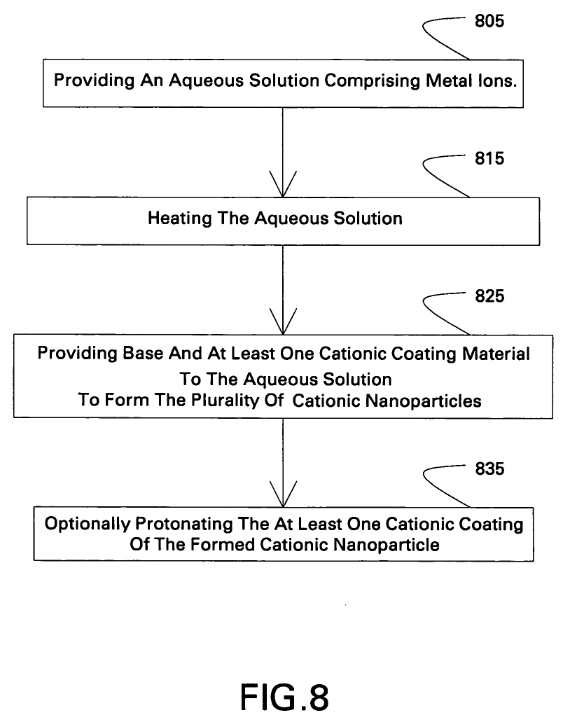

Another aspect of the invention is to provide a method of making a plurality of cationic nanoparticles as described hereinabove. FIG. 8 is a flow diagram of the method.

Referring to FIG. 8, step S805 comprises providing an aqueous solution comprising metal ions. An example of a source of the metal ions includes, but is not limited to, metal salts capable of forming the inorganic core 120. A particular source of the metal ions comprises a mixture of FeCl₂ and FeCl₃. In one embodiment, the ratio of Fe⁺³ to Fe⁺² is not greater than 2. In another embodiment, the amounts of FeCl₂ and FeCl₃ dissolved are selected to produce a Fe²⁺/Fe³⁺ molar ratio of 0.5.

In step S815, the aqueous solution is heated. For example, the aqueous solution may be heated to a temperature in range from about 30° C. to about 100° C.

In step S825, a base and at least one cationic coating material as described hereinabove are provided to the aqueous solution. The cationic coating material comprises at least one organo-silane, wherein the organo-silane comprises:
—Si(R¹)₃,
as previously described hereinabove and wherein at least one R¹ of the three R¹s is not an alkyl group.

Examples of bases include ammonium hydroxide and NaOH. The base reacts with the metal ions to form the inorganic core 120. The base also reacts with the at least one cationic coating material 140. The base provides a link between the inorganic core 120 and cationic coating material by catalyzing the hydrolysis and condensation reaction of the —Si(R¹)₃ organo-silane so that the cationic coating material substantially covers the inorganic core 120 to form the cationic nanoparticles 100.

The above steps are not limited by sequence. For example, the method is not limited by the sequence in which the aqueous solution comprising metal ions are provided and the aqueous solution is heated. Providing an aqueous solution comprising metal ions and heating the aqueous solution can be either simultaneously or sequentially performed. The method is also not limited by the sequence of providing the base and the cationic coating 140. The base and the cationic coating can either be sequentially or simultaneously provided. Furthermore, the method is also not limited by the sequence of providing an aqueous solution comprising metal ions, heating the aqueous solution, and providing the base and the cationic coating 140. Providing an aqueous solution comprising metal ions, heating the aqueous solution, and providing the base and cationic coating can be either simultaneously or sequentially performed.

In one embodiment, the method further includes step 835 of protonating the at least one outer cationic coating 140 of the formed cationic nanoparticle by adjusting the aqueous solution to a pH in a range from about 2 to about 9.

In a typical preparation, NaNO₃, FeCl₂ and FeCl₃.6H₂O are dissolved in deoxygenated water with vigorous stirring under nitrogen. The amount of FeC₁₂ and FeCl₃ dissolved are selected to produce a Fe²⁺/Fe³⁺ molar ratio of 0.5. The solution is heated to a temperature in a range from about 80° C. to about 90° C., and then charged by rapid addition of NH₄OH solution, an excess amount of coating material, and NaNO₃. Crystal growth is allowed to proceed for about 50 min at about 80° C. with constant, vigorous stirring to produce a stable colloidal suspension of nanoparticles. The aqueous suspension is then cooled slowly to room temperature with stirring. Once cooled, the suspension is allowed to sit atop a handheld magnet for about 8 hours to remove any insoluble material. Excess coating material is removed either by ultrafiltration or centrifugation. The final stable aqueous suspension, which is free of excess ligand, is sonicated in an ultrasonic bath for 1 hour and filtered.

The dimensions of the nanoparticles were characterized using the following techniques. Transmission electron microscopy (TEM) was used to determine the size of the inorganic cores 120 of the cationic nanoparticles 100. Dynamic light scattering (DLS) or photon correlation spectroscopy (PCS) was used to determine the hydrodynamic size of the cationic nanoparticles 100 in aqueous suspension. Magnetization was measured using a vibrating sample magnetometer with fields of up to 2,500 Gauss at 25° C. The relaxation times were measured by imaging nanoparticle suspensions at different concentrations at 25° C.

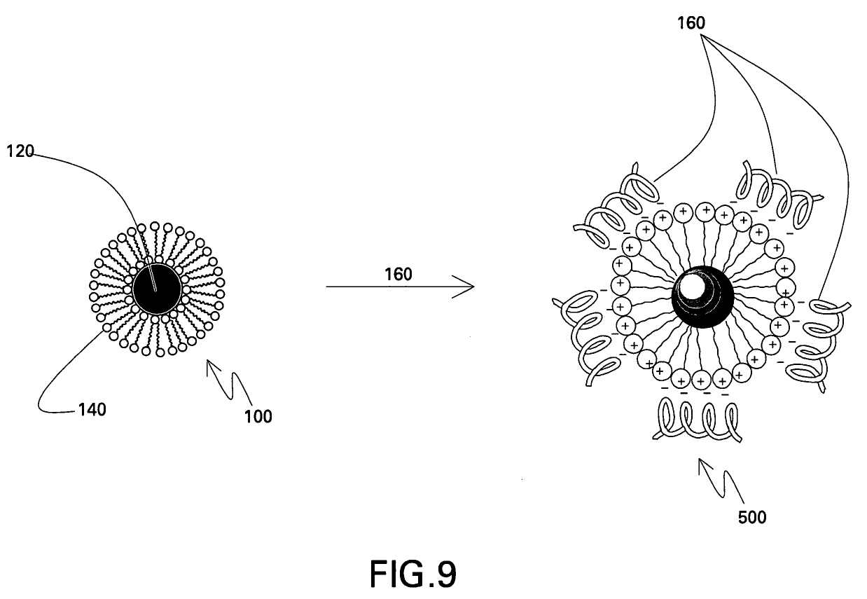

Another aspect of the invention is to provide a method of making a plurality of nanocomplexes 500 as previously described hereinabove. FIG. 9 is a schematic of the method. The method includes providing a plurality of oligonucleotides 160 and a plurality of cationic nanoparticles 100, as previously described hereinabove, into an aqueous solution. The method is not limited by the sequence in which the plurality of oligonucleotides 160 and the plurality of cationic nanoparticles 100 are provided to the aqueous solution. The plurality of oligonucleotides 160 and the plurality of cationic nanoparticles 100 can be either simultaneously or sequentially provided.

The method then involves attaching at least one oligonucleotides 160 to at least one cationic nanoparticle 100 to form the plurality of nanocomplexes. The oligonucleotides 160 may be attached in a variety of ways and orientations, as previously described hereinabove. The oligonucleotide 160 attaches to the cationic nanoparticle 100, as the negatively charged oligonucleotide 160 ionically interacts with the positively charged cationic coating 140.

The nanocomplexes 500 may have various uses. For example, the nanocomplexes 500 may be used in diagnostic medicine, molecular imaging, or as delivery agents or carriers. For example, the nanocomplexes may be used as agents for delivering oligonucleotides into cells in-vitro or into a subject in-vivo. Consequently, another aspect of the invention is to provide a method of delivering at least one oligonucleotide 160 into a cell. The method comprises providing at least one nanocomplex 500, as previously described hereinabove, into a solution containing a plurality of cells. The nanocomplex 500 is provided into the solution, which may comprise various cell types. In one embodiment, the cell type may be eukaryotic cell types, such as adherent, suspension, primary, and immortal cells, and may comprise any such individual cell types or any combination thereof. In one embodiment, the cell type comprises adherent rat macrophage.

In one example, the nanocomplex 500 is provided into a solution of cells by incubation, such as soaking the cell or organism in a solution comprising the nanocomplex 500. The nanocomplex 500 may also be provided into the solution by other methods, such as injection, bombardment by the nanocomplex 500, electroporation of cell membranes in the presence of the nanocomplex 500. Other methods known in the art for introducing oligonucleotides to cells may also be used, such as lipid-mediated carrier transport, chemical mediated transport, such as calcium phosphate, and the like. Thus, the nanocomplex 500 may be provided to the cells along with other components that perform one or more of the following activities: enhancing present oligonucleotide uptake by the cell; promoting annealing of the duplex strands, stabilizing the annealed strands; or otherwise increasing inhibition of a target gene.

When the nanocomplex 500 is provided into a solution of cells, the nanocomplex 500 may be provided into the cytoplasm of the cell, into the nucleus of the cell, or into of the organelles of the cell, such as the golgi apparatus, the endoplasmic reticulum, and mitochondria. Providing the nanocomplex 500 into solution may include providing the nanocomplex to any one of the sites mentioned above, or to any combination of such sites.

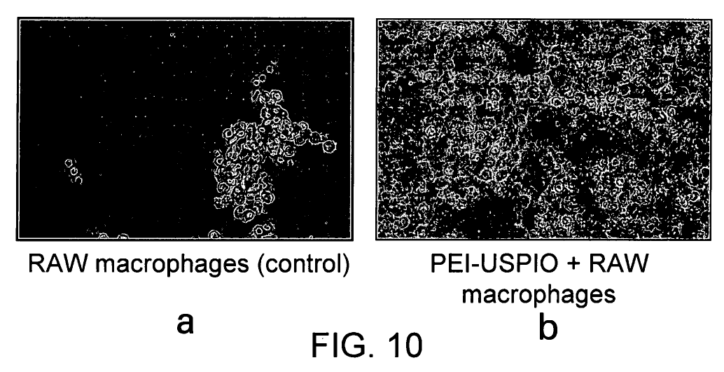

FIGS. 10a and 10b confirm the presence of nanocomplexes in the cells by showing the presence of iron oxide. FIG. 10a is a bright field microscopic image of cells fixed and Perl-stained that are not incubated with nanocomplexes 500. The cells are Mus musculus (mouse) macrophages with Designation RAW 264.7 gamma NO(−) and ATCC Number CRL-2278. A bright field microscopic image shows the presence of the iron oxide of the cationic nanoparticle 100, if present. In case of FIG. 10a, which is a control, the bright field microscopic image of the cells shows the absence of the nanocomplexes 500. FIG. 10b is a bright field microscopic image of cells after incubating the cells with nanocomplexes 500 for 24 hours. The FIG. 10b bright field microscopic image shows the presence of iron oxide in the nanocomplexes 500. Furthermore, because the iron oxide is in the same location as the cells shown in FIG. 10a, the FIG. 10b image shows that the iron oxide is located in the cells.

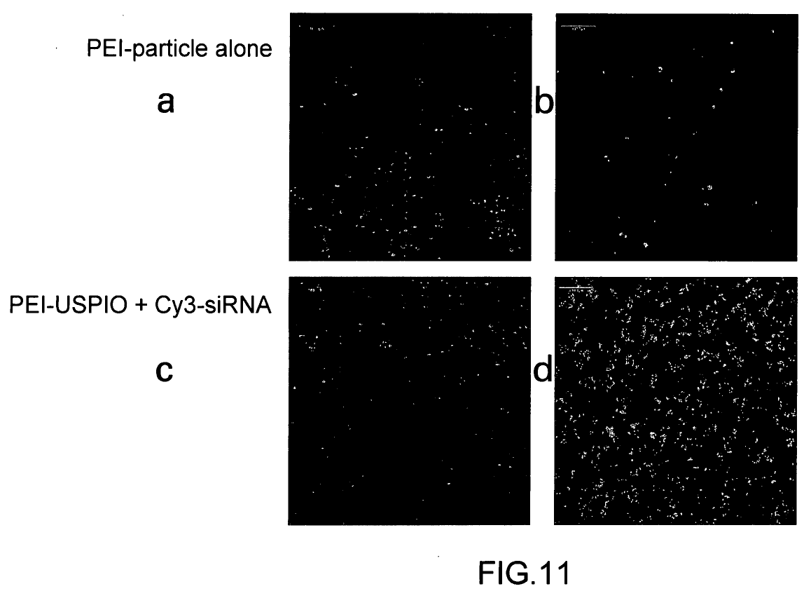

FIGS. 11a-11d confirm the presence of oligonucleotides in the cells. FIG. 11a and 11b are controls for demonstrating that incubating a cell with nanocomplexes 500 delivers oligonucleotides 16 into the cells. FIG. 11a is a bright field confocal microscopy image of cells after incubating for 6 hours with cationic nanoparticles 100 alone; i.e., nanoparticles that are not attached to an oligonucleotide 160. The cationic nanoparticles 100 comprise an organo-silane modified polyethyleneimine cationic coating. The cells are RAW mouse macrophages, as discussed previously. FIG. 11b is a laser excited fluorophore image of the same cells as shown in FIG. 11a. The laser excited fluorophore image shows the oligonucleotides, if present. In the case of FIG. 11b, which is a control, the FIG. 11b laser excited fluorophore images shows the absence of oligonucleotides 16.

FIG. 11c and 11d show that incubating a cell with nanocomplexes delivers oligonucleotides into the cells. FIG. 11c shows bright field confocal microscopy images of cells after incubating for 6 hours with nanocomplex 500 of cationic nanoparticles attached to an oligonucleotide. FIG. 11d are laser excited fluorophore confocal microscopy images of the same cells as shown in FIG. 11c. The FIG. 11d, laser excited fluorophore image shows the presence of oligonucleotides 16. Furthermore, because the oligonucleotides are in the same location as the cells shown in FIG. 11c, the FIG. 11d laser excited fluorophore image shows that the oligonucleotides 16 are present in the cells.

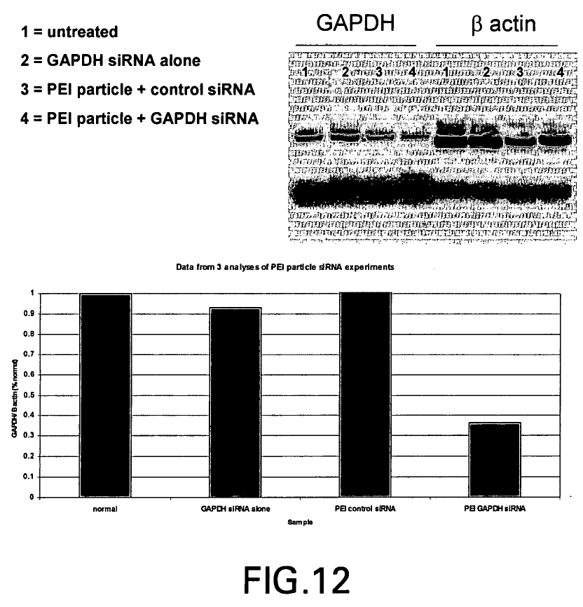

FIG. 12 shows that incubating cells with nanocomplexes delivers active oligonucleotides into cells, based on monitoring activity of decreased GADPH expression. “Active” means the oligonucleotides are capable of incorporation into RNA induced silencing complex for gene silencing in cytoplasm. Monitoring activity of decreased GADPH expression demonstrates delivery of active oligonucleotides into cells because decreased GADPH expression occurs if active oligonucleotides are delivered into cells. FIG. 12 shows RT-PCR analysis of mouse GAPDH and β actin mRNA after treatment of RAW mouse macrophages with nanocomplex 500 of cationic nanoparticles 100, using either control siRNA duplex (lane 3) or GAPDH-specific siRNA (lane 4) duplex. An untreated control is indicated in lane 1 and treatment of cells with GAPDH-specific siRNA duplex alone is indicated in lane 2.

Another aspect of the invention is to provide a method of delivering at least one oligonucleotide 160 to a subject. The method comprises administering at least one nanocomplex 500, as described hereinabove, to the subject.

Examples of such subjects include mammals, such as, but not limited to, rats, pigs, human, and the like. Administering the nanocomplex 500 may be accomplished orally, topically, parenterally, by inhalation spray, rectally, by subcutaneous injection, intravenous injection, intramuscular injection, intrasternal injection, infusion, and may comprise any such means individually or any combination thereof.

Another aspect of the invention is to provide a method of monitoring the delivery of at least one oligonucleotide 160 to a subject. The method comprises administering at least one nanocomplex 500, as described hereinabove, to a subject. A magnetic resonance image of the subject is obtained. The signal is correlated to the concentration of the oligonucleotide administered to the subject.

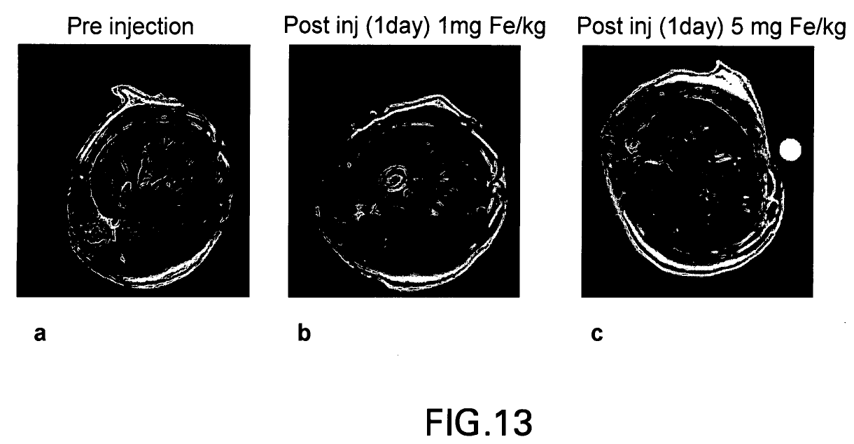

The delivery of oligonucleotides to a subject is illustrated in FIG. 13a-13c. FIG. 13a shows an in-vivo magnetic resonance image (MRI) of rat liver that has not been injected with a nanocomplex 500. FIG. 13b shows an in-vivo MRI of rat liver 24-hours after being injected with a nanocomplex to achieve 1 mg Fe/kg body weight. The cationic nanoparticles comprise an organo-silane modified polyethyleneimine cationic coating and are attached to siRNA duplex. The MRI (FIG. 13b) of the rat liver after injection shows greater contrast than the MRI shown in FIG. 13a. FIG. 13c shows an in-vivo MRI of rat liver 24 hours after being injected with a nanocomplex to achieve 5 mg Fe/kg body weight. Darkening is indicative of accumulation of nanoparticles in the liver. This MRI (FIG. 13c) of the rat liver shows even greater contrast than the MRI of the rat liver shown in FIG. 13a. Thus, the nanocomplex 500 might also be used as MRI contrast agents.

In one embodiment, attaching mature duplex siRNA to cationic metal oxide nanoparticles involves the following:

Using a synthetic duplex siRNA, an optimal concentration of siRNA is incubated with defined injectable dose of nanoparticle suspended in an aqueous buffer in a sterile container. After allowing the siRNA:nanoparticle nanocomplex to form between 5 and 30 minutes at room temperature, siRNA is delivered by one of the following methods: 1) adding the nanocomplex to a sterile cell culture containing cells of interest; 2) injecting the nanocomplex intravenously into animal using an allowed injection volume; 3) transfusing the nanocomplex intravenously into animals using allowed flow rates and injection volumes; 4) aerosolizing the nanocomplex and delivering it via inhalation; or 5) directly injecting the nanocomplex into a tissue or organ of interest. In one example, MRI could then be used to image the region of the organism where the nanoparticle complex is localized and validate that siRNA was delivered to the desired site.

An example of attaching other oligonucleotides, such as short hairpin RNA (shRNA) or vectors encoding short hairpin RNA, to cationic nanoparticles involves steps similar to those described for attaching the mature duplex siRNA to cationic nanoparticles, respectively.

An example of the delivery of vectors encoding short hairpin RNA and reporter gene constructs (including, but not limited to, luciferase-encoding vectors) using cationic metal oxide nanoparticles may involve the following:

Using a shRNA-coding vector and reporter gene construct, incubate optimal concentration vectors separately with defined injectable dose of nanoparticle suspended in an aqueous buffer in a sterile container. After allowing vector:nanoparticle complexes to form between 5 and 30 minutes at room temperature, deliver reporter vector in one of following methods: 1) adding complex to sterile cell culture containing cells of interest; 2) injecting IV into animal using allowed injection volume; 3) transfusing IV into animals using allowed flow rates and injection volumes; 4) aerosolizing complex and delivery via inhalation; or 5) directly injecting complex into tissue or organ of interest. In one example, MRI could then be used to image where the nanoparticle complex is localized and validate that vector was delivered to desired site. After allowing nanoparticle to clear from delivery site, deliver vector encoding shRNA in one of following methods: 1) add complex to sterile cell culture containing cells of interest or 2) inject IV into animal using allowed injection volume or 3) transfuse IV into animals using allowed flow rate and injection volume or 4) aerosolize complex and delivery via inhalation or 5) directly inject complex into tissue or organ of interest. Use optimal system to interrogate reporter gene activity before and after injection of vector encoding shRNA. Optimal systems may include charge coupled device or PET/CT imaging system. Efficiency of silencing activity of vector encoding shRNA may be quantified by reporter gene activity.

An example of delivering combinations of other oligonucleotides, such as short hairpin RNA and reporter gene (including but not limited to luciferase-encoding vectors) using cationic nanoparticles would involve steps similar to those described for delivering vectors encoding short hairpin RNA and reporter gene constructs.

The following examples serve to illustrate the features and advantages of the present invention and are not intended to limit the invention thereto.

### EXAMPLE 1

This example describes the preparation of N-trimethoxysilylpropyl-N,N,N,-tri-methylammonium coated magnetic nanoparticles. FeCl₂ and FeCl₃.6H₂O were dissolved in deoxygenated water with vigorous stirring under a nitrogen atmosphere. The amount of FeCl₂ and FeCl₃ that were dissolved were selected to produce a Fe²⁺/Fe³⁺ molar ratio of 0.5. The solution was heated to 85° C., and then charged by rapid addition of NH₄OH solution and N-trimethoxysilylpropyl-N,N,N,-tri-methylammonium chloride. The resulting reaction was allowed to proceed for about 1 hour at 85° C. with constant vigorous stirring to produce a stable colloidal solution of mixed Fe oxide nanoparticles. The solution was then cooled slowly to room temperature with stirring. Once cooled, the suspension was allowed to sit atop a handheld magnet for about 8 hr to remove any insoluble material. Excess N-trimethoxysilylpropyl-N,N,N,-tri-methylammonium chloride was removed by ultrafiltration. The final stable aqueous suspension, which was free of excess ligand, was sonicated in an ultrasonic bath for 1 hour and filtered. No color change was observed before and after filtration, indicating that there was no significant loss of iron oxide, which in turn suggests that there was no significant amount of agglomerated nanoparticles that were larger than the cut-off size of the filter. A cationic nanoparticle size of 16 nm was measured by dynamic light scattering (DLS).

### EXAMPLE 2

This example describes the preparation of N-[3-methoxysilyl)propyl]polyethyleneimine hydrochloride coated magnetic nanoparticles. NaNO₃, FeCl₂ and FeCl₃.6H₂O were dissolved in deoxygenated water with vigorous stirring under nitrogen. The amount of FeCl₂ and FeCl₃ that were dissolved were selected to produce a Fe²⁺/Fe³⁺ molar ratio of 0.5. The solution was heated to 80° C., and then charged by rapid addition of NH₄OH solution and N-[3-methoxysilylpropyl]polyethyleneimine hydrochloride. The resulting reaction was allowed to proceed for about 30 minutes at 80° C., and was then cooled slowly to room temperature with stirring. A dark brown solution was separated from a black precipitate. The black precipitate was re-suspended in water by adjusting the pH to a value in the range of 4-2 by adding aqueous HCl. Excess N-[3-methoxysilylpropyl]polyethyleneimine was removed by ultrafiltration. The final stable aqueous suspension, which was free of excess ligand, was sonicated in an ultrasonic bath for 1 hour and filtered. No color change was observed before and after filtration, indicating that there was no significant loss of iron oxide, which in turn suggests that there was no significant amount of agglomerated nanoparticles that were larger than the cut-off size of the filter. A cationic nanoparticle size of 15 nm was measured by DLS.

### EXAMPLE 3

This example describes the preparation of a nanocomplex comprising duplex siRNA. Cationic nanoparticles were filtered using sterile filters before incubating with duplex siRNA. After filtering, cationic nanoparticles were incubated at room temperature with sterile, labeled siRNA for at least 10 minutes under a sterile hood, wherein the duplex siRNA attached to the cationic nanoparticle 100. The nanocomplexes were then used for either gel-based siRNA binding assay or addition to cultures of cells in complete media.

For the gel-based siRNA-binding assay, agarose gels were prepared using water and agarose. Gels were cast in a pre-cleaned gel box to control for RNAses, which are proteins that degrade RNA. Samples containing siRNA alone, nanocomplexes of siRNA and iron oxide particles, or iron oxide particles alone were loaded in a mixture of glycerol and a gel was run. The gels were both scanned on an imager to detect Cy3 dye and stained for iron content using freshly prepared Perl stain solution. Gels were incubated in 100 mL of Perl stain for 15 minutes then photographed.

### EXAMPLE 4

This example describes the preparation of in-vitro delivery of GAPDH siRNA. RAW 264.7 mouse macrophages (ATCC) were cultured in complete media to >80% confluence culture plates. Nanocomplex mixtures comprising cationic particles attached to siRNA were added to wells to final concentrations of 500 nM iron oxide ±100 nM duplex siRNA against GAPDH. As an additional control, duplex siRNA (Ambion), which does not target GAPDH, was also complexed with the iron oxide nanoparticles at the concentrations indicated above and added to a culture of macrophages. Cultures were incubated under standard culture conditions for 48 hours and checked every 12 hours for microbial contamination. Following incubation, the total RNA from macrophage cultures was isolated and stored in nuclease-free water at −20° C.

Cy3-labeled siRNA against GAPDH was prepared as described above, added to the culture of TBP-1 monocytes, and incubated for 6 hours. Cells were then centrifuged and washed twice with sterile buffer. Cells were resuspended in sterile buffer and immediately viewed under confocal microscope to visualize Cy3 siRNA-iron oxide nanocomplexes interacting with THP-1 cells.

Reverse transcription (RT) polymerase chain reaction (PCR) analysis for mouse GAPDH action expression was performed using 250 ng of RNA per sample and oligo (dT)₁₅ primers. PCR was performed for GAPDH (Stratagene) and β actin (BD Clontech). Following 25 cycles of PCR, 10 μl of PCR mixture were loaded in agarose gel. Gels were run at 100V for 45 minutes and scanned for specific amplimers of GAPDH and β actin. Signal intensity was measured and plotted as a ratio of GAPDH/β actin band intensity.

### EXAMPLE 5

This example describes the preparation of administration of the nanocomplex to a subject in-vivo. Animals were scanned by magnetic resonance imaging to generate “pre-injection” T2-weighted MR images of a rat anatomy (FIG. 13a). A specific region of interest (ROI) was the liver. Sterile nanocomplex was then administered via tail vein injection to female Sprague-Dawley rats at dose of 1 mg Fe/kg body weight or 5 mg Fe/kg body weight in a total injection volume of 600 microliters.

### EXAMPLE 6

This example describes the preparation of monitoring the nanocomplex in-vivo. Following initial administration of nanocomplex, animals were transferred to cages for 24 hours and then imaged again to generate “post-injection” T2-weighted MR images of a rat anatomy. Again, the liver was identified as a region of interest (ROI) and several images were obtained. The decrease in signal intensity observed at 1 mg Fe and 5 mg Fe doses in liver (FIGS. 13b and 13c) are directly attributable to accumulation of the nanocomplex in liver.

While typical embodiments have been set forth for the purpose of illustration, the foregoing description should not be deemed to be a limitation on the scope of the invention. Accordingly, various modifications, adaptations, and alternatives may occur to one skilled in the art without departing from the spirit and scope of the present invention.

What is claimed is:

1. A cationic nanoparticle comprising:
(a) an inorganic core; and
(b) at least one outer cationic coating substantially covering the inorganic core, the at least one outer cationic coating comprising at least one organo-silane, wherein the at least one organo-silane comprises:
—Si(R¹)₃
wherein R¹ independently at each occurrence is an alkoxy group, a hydroxyl group, a halide, an alkyl group, or hydrogen, and wherein at least one R¹ of the three R¹s is not an alkyl group.

2. The cationic nanoparticle of claim 1, wherein the inorganic core is substantially monodisperse.

3. The cationic nanoparticle of claim 1, wherein the inorganic core is substantially crystalline.

4. The cationic nanoparticle of claim 1, wherein the cationic nanoparticle is substantially unagglomerated and has a diameter in a range from about 1 nm to about 100 nm.

5. The cationic nanoparticle of claim 4, wherein the cationic nanoparticle has a diameter in a range from about 5 nm to about 60 nm.

6. The cationic nanoparticle of claim 5, wherein the cationic nanoparticle has a diameter in a range from about 5 nm to about 20 nm.

7. The cationic nanoparticle of claim 1, wherein the at least one outer cationic coating comprises at least one of an organo-silane modified polyethylenimine, an organo-silane modified a poly(lysine), an organo-silane modified poly(aspargine), an organo-silane modified chitosane, an organo-silane modified poly(L-ornithine), an organo-silane modified poly(vinylamine), an organo-silane modified poly(amido amine), N-(trimethoxysilylethyl)benzyl-N,N,N-trimethylammonium chloride, an aminopropylsilanetriol, and combinations thereof.

8. The cationic nanoparticle of claim 7, wherein the at least one outer cationic coating comprises N-trimethoxysilylpropyl-N,N,N,tri-methylammonium salt.

9. The cationic nanoparticle of claim 7, wherein the at least one outer cationic coating comprises an organo-silane modified polyethylenimine.

10. The cationic nanoparticle of claim 9, wherein the at least one organo-silane —Si(R¹)₃ comprises trimethoxysilyl.

11. The cationic nanoparticle of claim 10, wherein the organo-silane modified polyethyleneimine has a molecular weight up to about 25,000 Da.

12. The cationic nanoparticle of claim 11, wherein the organo-silane modified polyethyleneimine has a molecular weight up to about 2,000 Da.

13. The cationic nanoparticle of claim 10, wherein the at least one organo-silane comprises from about 10% to about 60% by weight of the at least one outer cationic coating.

14. The cationic nanoparticle of claim 13, wherein the at least one organo-silane comprises from about 10% to 40% by weight of the at least one outer cationic coating.

15. The cationic nanoparticle of claim 14, wherein the at least one organo-silane comprises about 10% by weight of the at least one outer cationic coating.

16. The cationic nanoparticle of claim 1, wherein the at least one outer cationic coating comprises a plurality of organo-silanes.

17. The cationic nanoparticle of claim 1, further comprising at least one oligonucleotide attached to the cationic nanoparticle.

18. The cationic nanoparticle of claim 17, wherein the at least one oligonucleotide comprises at least one of a DNA molecule, a RNA molecule and combinations thereof.

19. The cationic nanoparticle of claim 18, wherein the at least one oligonucleotide comprises RNA.

20. The cationic nanoparticle of claim 19, wherein the RNA comprises at least one of a short inhibitory RNA, a short hairpin RNA, a micro RNA, and combinations thereof.

21. The cationic nanoparticle of claim 20, wherein the RNA comprises short inhibitory RNA.

22. The cationic nanoparticle of claim 21, wherein the short inhibitory RNA comprises up to about 100 base pairs.

23. The cationic nanoparticle of claim 22, wherein the short inhibitory RNA comprises up to about 40 base pairs.

24. The cationic nanoparticle of claim 23, wherein the short inhibitory RNA comprises up to about 24 base pairs.

25. A nanocomplex comprising:
(A) a cationic nanoparticle, the cationic nanoparticle comprising:
(a) an inorganic core; and
(b) at least one outer cationic coating substantially covering the inorganic core, the at least one outer cationic coating comprising at least one organo-silane, wherein the at least one organo-silane comprises:
—Si(R¹)₃
wherein R¹ independently at each occurrence comprises an alkoxy group, a hydroxyl group, a halide, an alkyl group, or hydrogen, and wherein at least one R¹ of the three R¹s is not an alkyl group; and
(B) at least one oligonucleotide attached to the cationic nanoparticle; and
wherein the nanocomplex is substantially unagglomerated.

26. The nanocomplex of claim 25, wherein the nanocomplex has a diameter in a range from about 1 nm to about 100 nm.

27. The nanocomplex of claim 26, wherein the nanocomplex has a diameter in a range from about 5 nm to about 60 nm.

28. The nanocomplex of claim 27, wherein the nanocomplex has a diameter in a range from about 5 nm to about 20 nm.

29. The nanocomplex of claim 25, wherein the at least one outer cationic coating comprises at least one of an organo-silane modified polyethylenimine, an organo-silane modified a poly(lysine), an organo-silane modified poly(aspargine), an organo-silane modified chitosane, an organo-silane modified poly(L-ornithine), an organo-silane modified poly(vinylamine), an organo-silane modified poly(amido amine), N-(trimethoxysilylethyl)benzyl-N,N,N-trimethylammonium chloride, an aminopropylsilanetriol, and combinations thereof.

30. The nanocomplex of claim 26, wherein the at least one outer cationic coating comprises an organo-silane modified polyethylenimine.

31. The nanocomplex of claim 30, wherein the at least one organo-silane —Si(R¹)₃ comprises trimethoxysilyl.

32. The nanocomplex of claim 31, wherein the organo-silane modified polyethyleneimine has a molecular weight up to about 25,000 Da.

33. The nanocomplex of claim 32, wherein the organo-silane modified polyethyleneimine has a molecular weight up to about 2,000 Da.

34. The nanocomplex of claim 31, wherein the at least one organo-silane comprises from about 10% to about 60% by weight of the at least one outer cationic coating.

35. The nanocomplex of claim 34, wherein the at least one organo-silane comprises from about 10% to about 40% by weight of the at least one outer cationic coating.

36. The nanocomplex of claim 35, wherein the at least one organo-silane comprises about 10% by weight of the at least one outer cationic coating.

37. The nanocomplex of claim 25, wherein the at least one outer cationic coating comprises a plurality of organo-silanes.

38. The nanocomplex of claim 25, wherein the at least one outer cationic coating comprises N-trimethoxysilylpropyl-N,N,N,-tri-methylammonium salt.

39. The nanocomplex of claim 25, wherein the at least one oligonucleotide comprises at least one of a DNA, RNA, and combinations thereof.

40. The nanocomplex of claim 39, wherein the at least one oligonucleotide comprises RNA.

41. The nanocomplex of claim 40, wherein the RNA comprises at least one of a short inhibitory RNA, a short hairpin RNA, a micro RNA, and combinations thereof.

42. The nanocomplex of claim 41, wherein the RNA comprises short inhibitory RNA.

43. The nanocomplex of claim 42, wherein the short inhibitory RNA comprises up to about 100 base pairs.

44. The nanocomplex of claim 43, wherein the short inhibitory RNA comprises up to about 40 base pairs.

45. The nanocomplex of claim 44, wherein the short inhibitory RNA comprises up to about 24 base pairs.

46. A method of making a plurality of cationic nanoparticles, wherein each cationic nanoparticle comprises:
(a) an inorganic core; and
(b) at least one outer cationic coating substantially covering the inorganic core, the at least one outer cationic coating comprising at least one organo-silane, wherein the at least one organo-silane comprises:
—Si(R¹)₃
wherein R¹ independently at each occurrence comprises an alkoxy group, a hydroxyl group, a halide, an alkyl group, or hydrogen, and wherein at least one R¹ of the three R¹s is not an alkyl group;
the method comprising the steps of:
(i) providing an aqueous solution comprising metal ions;
(ii) heating the aqueous solution;
(iii) providing a base and at least one cationic coating material to the aqueous solution, wherein the at least one cationic coating material comprises at least one organo-silane, wherein the at least one organo-silane comprises:
—Si(R¹)₃
wherein R¹ independently at each occurrence comprises an alkoxy group, a hydroxyl group, a halide, an alkyl group, or hydrogen, and wherein at least one R¹ of the three R¹s is not an alkyl group, and
wherein the base reacts with the metal ions to form the inorganic core and wherein the base reacts with the at least one cationic coating material to substantially cover the inorganic core to form the plurality of cationic nanoparticles; and
(v) optionally protonating the at least one outer cationic coating of the formed cationic nanoparticle by adjusting the aqueous solution to a pH in a range from about 2 to about 9.

47. The method of claim 46, wherein a source of the metal ions comprises metal salts capable of forming the inorganic core.

48. The method of claim 47, wherein the source of the metal ions comprises FeCl₂ and FeCl₃.

49. The method of claim 48, wherein the ratio of Fe⁺³ to Fe⁺² is not greater than 2.

50. The method of claim 46, wherein the inorganic core is magnetic.

51. The method of claim 50, wherein the inorganic core comprises iron oxide.

52. The method of claim 51, wherein the iron oxide comprises at least one of a magnetite, maghemite, and combinations thereof.

53. The method of claim 50, wherein the inorganic core is superparamagnetic.

54. The method of claim 46, wherein the cationic nanoparticle has a diameter in a range from about 5 nm to about 100 nm.

55. The method of claim 46, wherein the at least one outer cationic coating comprises at least one of an organo-silane modified polyethylenimine, an organo-silane modified a poly(lysine), an organo-silane modified poly(aspargine), an organo-silane modified chitosane, an organo-silane modified poly(L-omithine), an organo-silane modified poly(vinylamine), an organo-silane modified poly(amido amine), N-(trimethoxysilylethyl)benzyl-N,N,N-trimethylammonium chloride, an aminopropylsilanetriol, and combinations thereof.

56. The method of claim 46, wherein the at least one outer cationic coating comprises N-trimethoxysilylpropyl-N,N,N,-tri-methylammonium salt.

57. The method of claim 46, wherein at least one outer cationic coating comprises an organo-silane modified polyethylenimine.

58. The method of claim 57, wherein the at least one organo-silane —Si(R¹)₃ comprises trimethoxysilyl.

59. The method of claim 58, wherein the organo-silane modified polyethyleneimine has a molecular weight up to about 25,000 Da.

60. The method of claim 59, wherein the organo-silane modified polyethyleneimine has a molecular weight up to about 2,000 Da.

61. The method of claim 58, wherein the at least one organo-silane comprises from about 10% to about 40% by weight of the at least one outer cationic coating.

62. The method of claim 61, wherein the at least one organo-silane comprises about 10% by weight of the at least one outer cationic coating.

63. The method of claim 46, wherein the step of heating the aqueous solution comprises heating the aqueous solution at a temperature in a range from about 30° C. to about 100° C.

64. The method of claim 46, wherein the at least one outer cationic coating comprises a plurality of the organo-silanes.

65. A method of making a plurality of nanocomplexes wherein each nanocomplex comprises:
(A) a cationic nanoparticle comprising:
(a) an inorganic core; and
(b) at least one outer cationic coating substantially covering the inorganic core, the at least one outer cationic coating comprising at least one organo-silane, wherein the at least one organo-silane comprises:
—Si(R¹)₃
wherein R¹ independently at each occurrence comprises an alkoxy group, a hydroxyl group, a halide, an alkyl group, or hydrogen, and wherein at least one R¹ of the three R¹s is not an alkyl group;
(B) at least one oligonucleotide attached to the cationic nanoparticle; and
wherein the nanocomplex is substantially unagglomerated;
the method comprising the steps of:
(i) providing a plurality of oligonucleotides and a plurality of cationic nanoparticles into an aqueous solution, wherein each cationic nanoparticle comprises:
(a) an inorganic core; and
(b) at least one outer cationic coating substantially covering the inorganic core, the at least one outer cationic coating comprising at least one organo-silane, wherein the at least one organo-silane comprises:
—Si(R¹)₃
wherein R¹ independently at each occurrence comprises an alkoxy group, a hydroxyl group, a halide, an alkyl group, or hydrogen, and wherein at least one R¹ of the three R¹s is not an alkyl group;
(ii) attaching the at least one oligonucleotide to the at least one cationic nanoparticle, to form the plurality of the nanocomplexes.

66. The method of claim 65, wherein the nanocomplex has a diameter in a range from about 5 nm to about 100 nm.

67. The method of claim 65, wherein the at least one outer cationic coating comprises at least one of an organo-silane modified polyethylenimine, an organo-silane modified a poly(lysine), an organo-silane modified poly(aspargine), an organo-silane modified chitosane, an organo-silane modified poly(L-ornithine), an organo-silane modified poly(vinylamine), an organo-silane modified poly(amido amine), N-(trimethoxysilylethyl)benzyl-N,N,N-trimethylammonium chloride, an aminopropylsilanetriol, and combinations thereof.

68. The method of claim 67, wherein the at least one outer cationic coating comprises N-trimethoxysilylpropyl-N,N,N,-tri-methylammonium salt.

69. The method of claim 67, wherein the at least one cationic coating comprises an organo-silane modified polyethylenimine.

70. The method of claim 69, wherein the at least one organo-silane —Si(R¹)₃ comprises trimethoxysilyl.

71. The method of claim 70, wherein the organo-silane modified polyethyleneimine has a molecular weight up about 25,000 Da.

72. The method of claim 70, wherein the at least one organo-silane comprises from about 10% to about 60% by weight of the at least one outer cationic coating.

73. The method of claim 72, wherein the at least one organo-silane comprises from about 10% to about 40% by weight of the at least one outer cationic coating.

74. The method of claim 73, wherein the at least one organo-silane comprises about 10% by weight of the at least one outer cationic coating.

75. The method of claim 65, wherein the step of providing a plurality of cationic nanoparticles comprises providing sterile cationic nanoparticles.

76. The method of claim 65, wherein the at least one outer cationic coating comprises a plurality of the at least one organo-silanes.

77. The method of claim 65, wherein the plurality of oligonucleotides comprise at least one of a DNA, a RNA, and combinations thereof.

78. The method of claim 77, wherein the plurality of oligonucleotides comprise RNA.

79. The method of claim 78, wherein the RNA comprises at least one of a short inhibitory RNA, a short hairpin RNA, a micro RNA, and combinations thereof.

80. The method of claim 79, wherein the RNA comprises short inhibitory RNA.

81. The method of claim 80, wherein the short inhibitory RNA comprises up to about 100 base pairs.

82. The method of claim 65, wherein the step of attaching the at least one oligonucleotide to the at least one cationic nanoparticle comprises ionic interaction.

83. The method of claim 65, wherein the step of attaching the at least one oligonucleotide to the at least one cationic nanoparticle comprises incubating the at least one oligonucleotide and the at least one cationic nanoparticle.

84. A method of delivering at least one oligonucleotide into a cell, the method comprising the step of:
(i) providing at least one nanocomplex into a solution of cells, the at least one nanocomplex comprising:
(A) a cationic nanoparticle comprising:
(a) an inorganic core; and
(b) at least one outer cationic coating substantially covering the inorganic core, the at least one outer cationic coating comprising at least one organo-silane, wherein the at least one organo-silane comprises:
—Si(R¹)₃
wherein R¹ independently at each occurrence comprises an alkoxy group, a hydroxyl group, a halide, an alkyl group, or hydrogen, and wherein at least one R¹ of the three R¹s is not an alkyl group; and
(B) at least one oligonucleotide attached to the cationic nanoparticle;
and wherein the nanocomplex is substantially unagglomerated.

85. The method of claim 84, wherein the nanocomplex has a diameter in a range from about 5 nm to about 100 nm.

86. The method of claim 84, wherein the at least one outer cationic coating comprises at least one of an organo-silane modified polyethylenimine, an organo-silane modified a poly(lysine), an organo-silane modified poly(aspargine), an organo-silane modified chitosane, an organo-silane modified poly(L-ornithine), an organo-silane modified poly(vinylamine), an organo-silane modified poly(amido amine), N-(trimethoxysilylethyl)benzyl-N,N,N-trimethylammonium chloride, an aminopropylsilanetriol, and combinations thereof.

87. The method of claim 86, wherein the at least one outer cationic coating comprises N-trimethoxysilylpropyl-N,N,N,-tri-methylammonium salt.

88. The method of claim 86, wherein the at least one outer cationic coating comprises an organo-silane modified polyethylenimine.

89. The cationic nanoparticle of claim 88, wherein the at least one organo-silane —Si(R¹)₃ comprises trimethoxysilyl.

90. The method of claim 89, wherein the organo-silane modified polyethyleneimine has a molecular weight up to about 25,000 Da.

91. The method of claim 89, wherein the at least one organo-silane comprises from about 10% to about 60% by weight of the at least one outer cationic coating.

92. The method of claim 91, wherein the at least one organo-silane comprises from about 10% to about 40% by weight of the at least one outer cationic coating.

93. The method of claim 92, wherein the at least one organo-silane comprises about 10% by weight of the at least one outer cationic coating.

94. The method of claim 84, wherein the at least one outer cationic coating comprises a plurality of the at least one organo-silanes.

95. The method of claim 84, wherein the at least one oligonucleotide comprises at least one of a DNA, a RNA, and combinations thereof.

96. The method of claim 95, wherein the at least one oligonucleotide comprises RNA.

97. The method of claim 96, wherein the RNA comprises at least one of a short inhibitory RNA, a short hairpin RNA, a micro RNA, and combinations thereof.

98. The method of claim 97, wherein the RNA comprises short inhibitory RNA.

99. The method of claim 98, wherein the short inhibitory RNA comprises less than about 100 base pairs.

100. The method of claim 84, wherein the step of providing at least one nanocomplex into a solution of cells comprises incubating the at least one nanocomplex with the solution of cells.

101. The method of claim 84, wherein the step of providing at least one nanocomplex into a solution of cells comprises into at least one of a cytoplasm of the cells, an organelle of the cell, and any combinations thereof.

102. A method of delivering at least one oligonucleotide to a subject, the method comprising the step of:
(i) administering at least one nanocomplex to a subject, wherein the at least one nanocomplex comprises:
(A) a cationic nanoparticle comprising:
(a) an inorganic core; and
(b) at least one outer cationic coating substantially covering the inorganic core, the at least one outer cationic coating comprising at least one organo-silane, wherein the at least one organo-silane comprises:
—Si(R¹)₃
wherein R¹ independently at each occurrence is an alkoxy group, a hydroxyl group, a halide, an alkyl group, or hydrogen, and wherein at least one R¹ of the three R¹s is not an alkyl group; and
(B) at least one oligonucleotide attached to the cationic nanoparticle;
and wherein the nanocomplex is substantially unagglomerated.

103. The method of claim 102, wherein the step of administering the at least one nanocomplex comprises at least one of oral, topical, parenteral, inhalation spray, rectal, subcutaneous injection, intravenous injection, intramuscular injection, intrasternal injection, infusion, and combinations thereof.

104. The method of claim 102, wherein the nanocomplex has a diameter in a range from 5 nm to about 100 nm.

105. The method of claim 102, wherein the at least one outer cationic coating comprises N-trimethoxysilylpropyl-N,N,N,-tri-methylammonium salt.

106. The method of claim 102, wherein the at least one outer cationic coating comprises at least one of an organo-silane modified polyethylenimine, an organo-silane modified a poly(lysine), an organo-silane modified poly(aspargine), an organo-silane modified chitosane, an organo-silane modified poly(L-omithine), an organo-silane modified poly(vinylamine), an organo-silane modified poly(amido amine), N-(trimethoxysilylethyl)benzyl-N,N,N-trimethylammonium chloride, an aminopropylsilanetriol, and combinations thereof.

107. The method of claim 106, wherein the at least one outer cationic coating comprises an organo-silane modified polyethylenimine.

108. The cationic nanoparticle of claim 107, wherein the at least one organo-silane —Si(R¹)3 comprises trimethoxysilyl.

109. The method of claim 108, wherein the organo-silane modified polyethyleneimine has a molecular weight up to about 25,000 Da.

110. The method of claim 108, wherein the at least one organo-silane comprises from about 10% to about 60% by weight of the at least one outer cationic coating.

111. The method of claim 110, wherein the at least one organo-silane comprises from about 10% to about 40% by weight of the at least one outer cationic coating.

112. The method of claim 111, wherein the at least one organo-silane comprises about 10% by weight of the at least one outer cationic coating.

113. The method of claim 102, wherein the at least one outer cationic coating comprises a plurality of the at least one organo-silanes.

114. The method of claim 102, wherein the at least one oligonucleotide comprises at least one of a DNA, RNA, and combinations thereof.

115. The method of claim 114, wherein the at least one oligonucleotide comprises RNA.

116. The method of claim 115, wherein the RNA comprises at least one of a short inhibitory RNA, a short hairpin RNA, a micro RNA, and combinations thereof.

117. The method of claim 116, wherein the RNA comprises short inhibitory RNA.

118. The method of claim 117, wherein the short inhibitory RNA comprises less than about 100 base pairs.

119. The method of claim 118, wherein the short inhibitory RNA comprises less than about 40 base pairs.

120. The method of claim 119, wherein the short inhibitory RNA comprises less than about 24 base pairs.

121. A method of monitoring the delivery of at least one oligonucleotide to a subject, the method comprising the steps of:
(i) administering at least one nanocomplex to a subject, the at least one nanocomplex comprising:
(A) a cationic nanoparticle comprising:
(a) an inorganic core; and
(b) at least one outer cationic coating substantially covering the inorganic core, the at least one outer cationic coating comprising at least one organo-silane, wherein the at least one organo-silane comprises:
—Si(R¹)₃
wherein R¹ independently at each occurrence comprises an alkoxy group, a hydroxyl group, a halide, an alkyl group, or hydrogen, and wherein at least one R¹ of the three R¹s is not an alkyl group; and
(B) at least one oligonucleotide attached to the cationic nanoparticle;
and wherein the nanocomplex is substantially unagglomerated;
(ii) obtaining a magnetic resonance image of the subject to achieve a signal of the concentration of the at least one nanocomplex administered to the subject; and
(iii) correlating the signal of the at least one nanocomplex to the concentration of the at least one oligonucleotide administered to the subject.

122. The method of claim 121, wherein the step of administering at least one nanocomplex comprises at least one of oral, topical, parenteral, inhalation spray, rectal, subcutaneous injection, intravenous injection, intramuscular injection, intrasternal injection, infusion, and combinations thereof.

123. The method of claim 121, wherein the nanocomplex has a diameter in a range from about 20 nm to about 50 nm.

124. The method of claim 121, wherein the at least one outer cationic coating comprises N-trimethoxysilylpropyl-N,N,N,-tri-methylammonium salt.

125. The method of claim 121, wherein the at least one outer cationic coating comprises at least one of an organo-silane modified polyethylenimine, an organo-silane modified a poly(lysine), an organo-silane modified poly(aspargine), an organo-silane modified chitosane, an organo-silane modified poly(L-ornithine), an organo-silane modified poly(vinylamine), an organo-silane modified poly(amido amine), N-(trimethoxysilylethyl)benzyl-N,N,N-trimethylammonium chloride, an aminopropylsilanetriol, and combinations thereof.

126. The method of claim 125, wherein the at least one outer cationic coating comprises an organo-silane modified polyethylenimine.

127. The cationic nanoparticle of claim 126, wherein the at least one organo-silane —Si(RI)3 comprises trimethoxysilyl.

128. The method of claim 127, wherein the organo-silane modified polyethyleneimine has a molecular weight up to about 25,000 Da.

129. The method of claim 127, wherein the at least one organo-silane comprises from about 10% to about 60% by weight of the at least one outer cationic coating.

130. The method of claim 129, wherein the at least one organo-silane comprises from about 10% to about 40% by weight of the at least one outer cationic coating.

131. The method of claim 130, wherein the at least one organo-silane comprises about 10% by weight of the at least one outer cationic coating.

132. The method of claim 121, wherein the at least one outer cationic coating comprises a plurality of the at least one organo-silanes.

133. The method of claim 121, wherein the at least one oligonucleotide comprises at least one of a DNA, a RNA, and combinations thereof.

134. The method of claim 133, wherein the at least one oligonucleotide comprises RNA.

135. The method of claim 134, wherein the RNA comprises at least one of a short inhibitory RNA, a short hairpin RNA, a micro RNA, and combinations thereof.

136. The method of claim 135, wherein the RNA comprises short inhibitory RNA.

137. The method of claim 136, wherein the short inhibitory RNA comprises less than about 100 base pairs.

138. The method of claim 137, wherein the short inhibitory RNA comprises less than about 40 base pairs.

139. The method of claim 138, wherein the short inhibitory RNA comprises less than about 24 base pairs.

140. The method of claim 139, wherein the short inhibitory RNA comprises less than about 24 base pairs.
